(19) United States
(12) Patent Application Publication
(10) Pub. No.: US 2026/0294784 A1
(43) Pub. Date: Oct. 1, 2026

(54) COSMETIC COMPOSITIONS AND PRODUCTS THEREOF SUBSTANTIALLY FREE OF SIMPLE ALCOHOL AND WAX

(71) Applicant: SGII, INC. (DBA SENEGENCE), Foothill Ranch, CA (US)

(72) Inventors: Jenelle Marie MOAD, Santa Ana, CA (US); Chelsea KINCEL, Irvine, CA (US); Nikki CHEW, Tustin, CA (US)

(21) Appl. No.: 19/474,809

(22) PCT Filed: Apr. 11, 2024
(86) PCT No.: PCT/US24/23966

§ 371 Date: Oct. 13, 2025

Related U.S. Application Data
(60) Provisional application No. 63/495,726, filed on Apr. 12, 2023.

(51) Int. Cl. A61K 8/892; A61K 8/365; A61K 8/60; A61K 8/73; A61K 8/97; A61Q 1/04

(52) U.S. Cl. CPC A61K 8/892; A61K 8/365; A61K 8/60; A61K 8/73; A61K 8/97; A61Q 1/04; A61K 2800/30; A61K 2800/43; A61K 2800/87

(57) ABSTRACT

Cosmetic compositions, cosmetic products and methods and uses thereof are provided. The cosmetic composition comprising water, trisiloxane, trimethylsiloxysilicate, sodium hyaluronate, xylitol, androxylitol, xylityl glucosides, hexyl/succinyl dimethicone crosspolymer, at least one natural component, at least one coloring agent, and at least one additional component. Cosmetic compositions and products as described herein may be substantially free of simple alcohol and/or wax.

This application claims the priority benefit of U.S. Provisional Patent Application Ser. No. 63/495,726, filed Apr. 12, 2023, which is hereby incorporated by reference in its entirety.

### TECHNICAL FIELD

The present disclosure relates to cosmetic compositions and cosmetic products that are substantially free of wax and simple alcohols, particularly those to be applied to the lips, as well as methods and uses thereof.

### BACKGROUND OF THE INVENTION

Cosmetic compositions and products that do not contain wax and/or alcohol have become more desirable, as components, such as alcohols, may irritate or dry the skin, including the lips, of the user. While desiring cosmetics devoid of components which may cause discomfort, potential negative health consequences and/or derived from non-ethical sources, consumers still require a composition or product with similar properties as conventional wax and/or alcohol containing compositions and products, such properties including color, shine, finish, length of wear, ease of removal, resistance to smearing, resistance to flaking, waterproof, moisturizing ability, and/or ease of application, such as no need for additional subsequent products including, for example, lip gloss.

Cosmetic preparations are known to contain a variety of alcohols, such as, for example fatty alcohols, which include cetyl alcohol, cetearyl alcohol, and behenyl alcohol, and for example, simple alcohols including isopropanol, ethanol, methanol or denatured alcohol. Simple alcohols, including denatured alcohol, are a common ingredient in many cosmetics as they provide beneficial properties to such formulations, such as promoting absorption, improving the ability to spread the cosmetic, faster drying, and acting as a preservative. Prolonged exposure to simple alcohols in such cosmetics may have negative consequences such as skin dryness, stinging, discomfort, redness, irritation, pain and/or sensitivity. Therefore, alternative cosmetic products that do not contain simple alcohols are desired.

Additionally, many cosmetic compositions are formulated with wax, as waxes are solids at room temperature and therefore are able to provide structure to cosmetic compositions and products, particularly those to be applied to the lips. Cosmetic containing waxes are generally provided as mineral waxes derived from petroleum, such as paraffin or ozokerite or natural waxes such as beeswax or lanolin. Alternatives to the use of waxes are required, as consumers often seek out petroleum free products due to potential negative environmental and health consequences, and consumers seek out animal-product free, ethically sourced and/or vegan-certified products.

Certain cosmetic compositions and products, such as those for application to the lips such as lip-gloss, lip cream and lipstick require different considerations than cosmetic compositions and cosmetic products for application to other areas of the body as, in addition to appearance, these products are required to have both desirable smell and taste for the user. Moreover, as the user wears said cosmetics on their lips, the compositions and products need to be more resistant to smear, budging or inadvertent removal, as once applied, said cosmetics are exposed to conditions such as eating, speaking, and contact with the user's tongue, and as such, said cosmetics require unique properties to limit the negative impacts for the user. Waxes, such as for example lanolin, are often included in cosmetics intended for usage on a user's lips to provide thickness to the product and help the product or composition stay on the user's lips, and therefore providing wax-free cosmetics for application to the lips are required.

As simple alcohols and wax provide important features to cosmetics, alternatives are sought that maintain the benefits of such alcohol and/or wax containing products without the need for their inclusion in any compositions or products thereof. Simple alcohol and wax free cosmetic compositions and products thereof provided in a one-step formulation that maintains desirable properties such as texture, color, finish, length of wear, waterproof capability, comfort and moisturizing are therefore required.

### SUMMARY OF THE INVENTION

In an embodiment herein there is provided a cosmetic composition that may comprise water, trisiloxane, trimethylsiloxysilicate, sodium hyaluronate, xylitol, androxylitol, xylityl glucosides, hexyl/succinyl dimethicone crosspolymer, at least one natural component, at least one coloring agent, and at least one additional component.

In certain embodiments, the at least one natural component, the at least one coloring agent and the at least one additional component may not comprise a simple alcohol, a wax, or both.

In certain embodiments, the wax may be a mineral wax, a wax derived from an animal source, or a non-vegan wax.

In certain embodiments, the simple alcohol may be ethyl alcohol, isopropyl alcohol, methyl alcohol, benzyl alcohol, denatured alcohol, or any combination thereof.

In certain embodiments, the at least one natural component may comprise Pyrus malus fruit extract, Glycine soja oil, Butyrospermum parkii butter, Aloe barbadensis leaf juice, Calanthe discolor extract, Laminaria digitata extract, Saccharomyces cerevisiae extract, Camellia sinensis leaf extract, Coffea arabica seedcake extract, Euterpe oleracea fruit extract, Garcinia mangostana fruit extract, Lycium barbarum fruit extract, Morinda citrifolia fruit extract, Orchis mascula flower extract, Punica granatum fruit extract and/or hydrolyzed Opuntia ficus-indica flower extract.

In certain embodiments, the at least one coloring agent may comprise at least one white coloring agent, at least one red coloring agent, at least one blue coloring agent, at least one saffron coloring agent and at least one yellow coloring agent.

In certain embodiments, the at least one coloring agent may comprise: CI 10006, CI 10020, CI 10316, CI 11680, CI 11710, CI 11725, CI 11920, CI 12010, CI 12085, CI 12120, CI 12370, CI 12420, CI 12480, CI 12490, CI 12700, CI 13015, CI 14270, CI 14700, CI 14720, CI 14815, CI 15510, CI 15525, CI 15580, CI 15620, CI 15630, CI 15800, CI 15850, CI 15865, CI 15880, CI 15980, CI 15985, CI 16035, CI 16185, CI 16230, CI 16255, CI 16290, CI 17200, CI 18050, CI 18130, CI 18690, CI 18736, CI 18820, CI 18965, CI 19140, CI 20040, CI 20470, CI 21100, CI 21108, CI 21230, CI 24790, CI 26100, CI 27755, CI 28440, CI 40215, CI 40800, CI 40820, CI 40825, CI 40850, CI 42045, CI 42051, CI 42053, CI 42080, CI 42090, CI 42100, CI 42170, CI 42520, CI 42735, CI 44090, CI 45100, CI 45190, CI 45220, CI 45350, CI 45370, CI 45380, CI 45396, CI 45405, CI 45410, CI 45430, CI 47000, CI 47005, CI 50325, CI 50420, CI 51319, CI 58000, CI 59040, CI 60724, CI 60725, CI 60730, CI 61565, CI 61570, CI 61585, CI 62045, CI 69800, CI 69825, CI 71105, CI 73000, CI 73015, CI 73360, CI 73385, CI 73900, CI 73915, CI 74100, CI 74160, CI 74180, CI 74260, CI 75100, CI 75120, CI 75125, CI 75130, CI 75135, CI 75170, CI 75300, CI 75470, CI 75810, CI 77000, CI 77002, CI 77004, CI 77007, CI 77015, CI 77019, CI 77120, CI 77163, CI 77220, CI 77231, CI 77266, CI 77267, CI 77268:1, CI 77288, CI 77289, CI 77346, CI 77400, CI 77480, CI 77489, CI 77491, CI 77492, CI 77499, CI 77510, CI 77713, CI 77742, CI 77745, CI 77820, CI 77891, CI 77947, Lactoflavin, Caramel, Capsanthin, capsorubin, Beetroot red, Anthocyanins, Aluminum stearate, Zinc stearate, Magnesium stearate, Calcium stearate, Bromothymol blue, Bromocresol green, Acid red 195, any lakes and salts formed by the listed coloring agent, or any combination thereof.

In certain embodiments, the at least one coloring agent may comprise CI 42090, CI 15850, CI 19140, CI 77491 and CI 77891.

In certain embodiments, the at least one additional component may comprise: cyclopentasiloxane; isododecane; dimethicone crosspolymer; dimethicone; acrylates copolymer; PEG-10 dimethicone; disteardimonium hectorite; glycerin; sodium chloride; phenoxyethanol; polyhydroxystearic acid; silica; glucose; ethylhexylglycerin; aroma and flavor; tocopheryl acetate; panthenol; butylene glycol; urea; glucosamine HCL; tocopherol; citric acid; xanthan gum; gluconolactone; 1,2-hexanediol; caprylyl glycol; sodium benzoate; and/or calcium gluconate.

In certain embodiments there is provided herein a cosmetic composition comprising: water, isododecane, trisiloxane, trimethylsiloxysilicate, cyclopentasiloxane, dimethicone crosspolymer, hexyl/succinyl dimethicone crosspolymer, dimethicone, acrylates copolymer, PEG-10 dimethicone, disteardimonium hectorite, glycerin, Pyrus malus fruit extract, xylitylglucoside, sodium chloride, phenoxyethanol, anhydroxylitol, Glycine soja oil, xylitol, polyhydroxystearic acid, silica, glucose, Butyrospermum parkii butter, ethylhexylglycerin, aroma and flavor, Aloe barbadensis leaf juice, sodium hyaluronate, tocopheryl acetate, panthenol, butylene glycol, Calanthe discolor extract, urea, glucosamine HCl, Laminaria digitata extract, Saccharomyces cerevisiae extract, Camellia sinensis leaf extract, Coffea arabica seedcake extract, Euterpe oleracea fruit extract, Garcinia mangostana fruit extract, Lycium barbarum fruit extract, Morinda citrifolia fruit extract, Orchis mascula flower extract, Punica granatum fruit extract, tocopherol, hydrolyzed Opuntia ficus-indica flower extract, citric acid, xanthan gum, gluconolactone, 1,2-hexanediol, caprylyl glycol, sodium benzoate, calcium gluconate, and at least one coloring agent.

In certain embodiments there is provided herein a cosmetic composition comprising: water, isododecane, trisiloxane, trimethylsiloxysilicate, cyclopentasiloxane, dimethicone crosspolymer, hexyl/succinyl dimethicone crosspolymer, dimethicone, acrylates copolymer, CI 77891, PEG-10 dimethicone, CI 77491, disteardimonium hectorite, glycerin, Pyrus malus fruit extract, xylitylglucoside, sodium chloride, phenoxyethanol, anhydroxylitol, Glycine soja oil, xylitol, CI 19140, polyhydroxystearic acid, CI 15850, silica, glucose, Butyrospermum parkii butter, ethylhexylglycerin, CI 42090, aroma and flavor, Aloe barbadensis leaf juice, sodium hyaluronate, tocopheryl acetate, panthenol, butylene glycol, Calanthe discolor extract, urea, glucosamine HCl, Laminaria digitata extract, Saccharomyces cerevisiae extract, Camellia sinensis leaf extract, Coffea arabica seedcake extract, Euterpe oleracea fruit extract, Garcinia mangostana fruit extract, Lycium barbarum fruit extract, Morinda citrifolia fruit extract, Orchis mascula flower extract, Punica granatum fruit extract, tocopherol, hydrolyzed Opuntia ficus-indica flower extract, citric acid, xanthan gum, gluconolactone, 1,2-hexanediol, caprylyl glycol, sodium benzoate, and calcium gluconate.

In certain embodiments there is provided herein a cosmetic composition comprising:
(a) 10%-25% of each of water, isododecane and trisiloxane;
(b) 5%-10% of trimethylsiloxysilicate;
(c) 1%-5% of each of cyclopentasiloxane, dimethicone crosspolymer, hexyl/succinyl dimethicone crosspolymer, dimethicone, acrylates copolymer, PEG-10 dimethicone, disteardimonium hectorite, glycerin, Pyrus malus fruit extract, and xylitylglucoside;
(d) 0.1-1% of each of sodium chloride, phenoxyethanol, anhydroxylitol, Glycine soja oil, xylitol, polyhydroxystearic acid, silica and glucose;
(e) 0%> but ≤0.5% of each of: Butyrospermum parkii butter; ethylhexylglycerin; aroma and flavor; Aloe barbadensis leaf juice; sodium hyaluronate; tocopheryl acetate; panthenol; butylene glycol; Calanthe discolor extract; urea; glucosamine HCL; Laminaria digitata extract; Saccharomyces cerevisiae extract; Camellia sinensis leaf extract; Coffea arabica seedcake extract; Euterpe oleracea fruit extract; Garcinia mangostana fruit extract; Lycium barbarum fruit extract; Morinda citrifolia fruit extract; Orchis mascula flower extract; Punica granatum fruit extract; tocopherol; hydrolyzed Opuntia ficus-indica flower extract; citric acid; xanthan gum; gluconolactone; 1,2-hexanediol; caprylyl glycol; sodium benzoate; and calcium gluconate; and
(f) one or more coloring agent.

In certain embodiments there is provided herein a cosmetic composition consisting of:
(a) 10%-25% of each of water, isododecane and trisiloxane;
(b) 5%-10% of trimethylsiloxysilicate;
(c) 1%-5% of each of cyclopentasiloxane, dimethicone crosspolymer, hexyl/succinyl dimethicone crosspolymer, dimethicone, acrylates copolymer, CI 77891, PEG-10 dimethicone, CI 77491, disteardimonium hectorite, glycerin, Pyrus malus fruit extract, and xylitylglucoside;
(d) 0.1-1% of each of sodium chloride, phenoxyethanol, anhydroxylitol, Glycine soja oil, xylitol, CI 19140, polyhydroxystearic acid, CI 15850, silica and glucose; and
(e) 0%> but ≤0.5% of each of: Butyrospermum parkii butter; ethylhexylglycerin; CI 42090; aroma and flavor; Aloe barbadensis leaf juice; sodium hyaluronate; tocopheryl acetate; panthenol; butylene glycol; Calanthe discolor extract; urea; glucosamine HCL; Laminaria digitata extract; Saccharomyces cerevisiae extract; Camellia sinensis leaf extract; Coffea arabica seedcake extract; Euterpe oleracea fruit extract; Garcinia mangostana fruit extract; Lycium barbarum fruit extract; Morinda citrifolia fruit extract; Orchis mascula flower extract; Punica granatum fruit extract; tocopherol; hydrolyzed Opuntia ficus-indica flower extract; citric acid; xanthan gum; gluconolactone; 1,2-hexanediol; caprylyl glycol; sodium benzoate; and calcium gluconate.

In certain embodiments, the composition may be for use on a user's lips.

In certain embodiments, the composition may provide one or more of the following when applied to the user's lips: resistance to smearing, non-drying, moisturizing, no discomfort, creation of unique colors by layering the composition, waterproof, long-lasting, gluten free, animal by-product free, and paraben free.

In certain embodiments there is provided herein a cosmetic product comprising a composition as described herein.

In certain embodiments, product may be a lip cream, lip balm, lipstick or a lip-gloss.

In certain embodiments, the product may provide one or more of the following when applied to a user: resistance to smearing, non-drying, moisturizing, no discomfort, creation of unique colors by layering, waterproof, long-lasting, gluten free, animal by-product free, and paraben free.

In certain embodiments there is provided herein a use of a cosmetic composition as described herein or a cosmetic product as described herein for providing color to the lips of a user when the composition or product is applied.

In certain embodiments, the composition may be for a single application.

In certain embodiments, the composition may be for multiple applications.

In certain embodiments, the product, composition, or both may provide one or more of the following when applied to the user: resistance to smearing, non-drying, moisturizing, no discomfort, creation of unique colors by layering, waterproof, long-lasting, gluten free, animal by-product free, and paraben free.

In certain embodiments there is provided herein a method for obtaining a desired lip coloring and/or lip moisture comprising applying a composition as described herein or a product as described herein to a user's lips.

In certain embodiments, the composition or product may be applied to the user's lips one or more times to achieve the desired lip coloring and/or lip moisturization.

In certain embodiments, the composition or product may be long-lasting on the user's lips.

In certain embodiments, the composition or product may be resistant to transfer from the user's lips.

In certain embodiments, the composition or product may be waterproof on the user's lips.

In certain embodiments, the product, composition, or both may provide one or more of the following when applied to the user: resistance to smearing, non-drying, moisturizing, no discomfort, creation of unique colors by layering, waterproof, long-lasting, gluten free, no animal by-products, and paraben free.

In certain embodiments there is provided herein a composition, kit, method, use, and/or product as described herein.

### BRIEF DESCRIPTION OF THE DRAWINGS

These and other features of the invention will become more apparent from the following description in which reference is made to the appended drawings wherein:

FIG. 1 is a photographic image of a user's lips with a cosmetic product as described herein applied thereto;

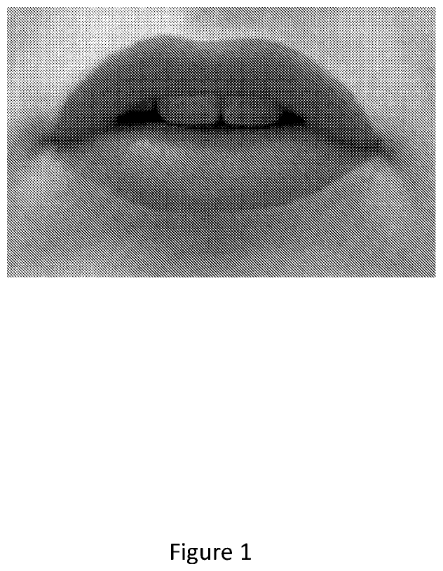

FIG. 2 is a photographic image of a user's lips with a cosmetic product as described herein applied thereto following the user eating with the applied cosmetic product. The applied product appears similar after eating to that observed in FIG. 1; and

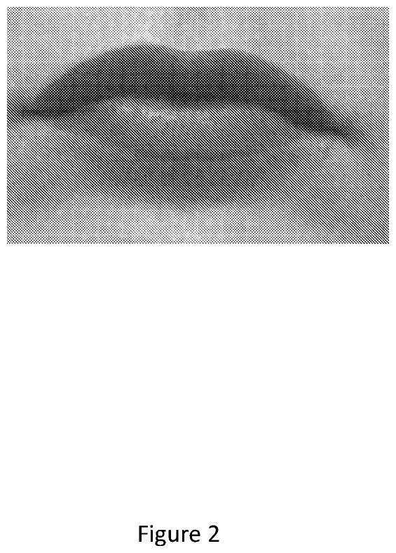

FIG. 3 is a photographic image of a user's fingers following the user rubbing their lips with the applied cosmetic product to determine the extent of smearing and transfer. Minimal transfer, as observed by the presence of only a few small patches (red) of product on the fingers of the user can be observed following the rubbing.

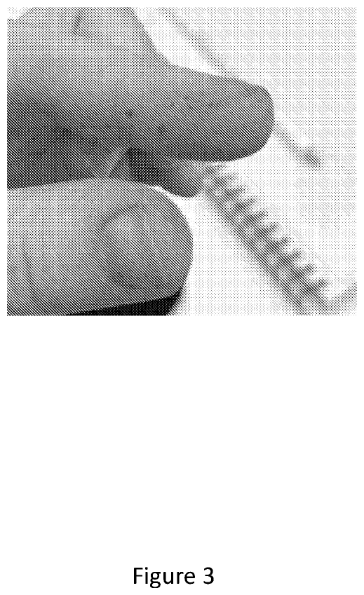

### DETAILED DESCRIPTION

Described herein are cosmetic compositions, products comprising cosmetic compositions, and methods and uses thereof. It will be appreciated that embodiments and examples are provided herein for illustrative purposes intended for those skilled in the art, and are not meant to be limiting in any way.

As used herein, a “cosmetic” may refer to a composition or product that is intended for application to a user's skin to enhance or protect the appearance of the user when worn, such as for example, the color, texture, feel, look, or any combination thereof. Examples of cosmetics may include, but are not limited to, foundation, eye shadow, eye liner, mascara, blush, and cosmetics intended for use on the lips. Cosmetics to be applied to a user's lips may include lip stick, lip gloss, lip cream, lip balm, lip color, or any combination or variant thereof.

Cosmetic compositions may be defined as compositions suitable for application to the human body. Cosmetic compositions such as creams and lotions may be used to moisturize the skin and keep the skin of the user smooth and hydrated. Pigmented cosmetic compositions such as makeup, blush, lipstick, lip cream, lip color, lip gloss, and eyeshadow, may be used to color the skin and lips. Additionally, pigmented creams and lotions may be used to moisturize and color the skin including the lips of the user. The skilled person would select components for a cosmetic composition or product as described herein that conform to local regulations provided for the manufacture and sale of cosmetics therein, such as for example, the Food and Drugs Act in the United States of America, Health Canada in Canada, the Industrial Chemicals Act in Australia, the Environmental Protection Authority (Cosmetic Products Group Standard) in New Zealand, the Cosmetic Supervision and Administration Regulation in China, or any equivalent international, national, local or non-governmental regulatory body.

In certain embodiments as described herein components of the composition may be referred to using names provided by the International Nomenclature of Cosmetic Ingredients (INCI), or any equivalent thereof. The skilled person, in light of the teachings herein, would be able to select the appropriate component using the INCI designation of the component or an equivalent thereof, such as for example, CI 77891 may be referred to as such or as titanium dioxide or pigment white. Furthermore, the skilled person, in light of the teachings herein, would be able to select an appropriate commercial equivalent of said component, such as for example Creasperse® ID White R to provide CI 77891.

As used herein a “simple alcohol” may refer to an alcohol derived from the fermentation of sugars, starches, and/or carbohydrates. In certain embodiments, the product or composition may be substantially free or devoid of alcohol or simple alcohols. In certain embodiments, the composition or product may comprise one of more alcohols known to the skilled person to not have a negative effect on the skin to which it is applied, such as, for example, fatty alcohols. In certain embodiments, the negative effects on the skin to which the composition or product are applied may comprise discomfort including pain or stinging, drying of the users skin, drying of the product or composition when worn, redness, irritation, or any combination thereof. In certain embodiments, the simple alcohol may comprise one or more of ethyl alcohol, isopropyl alcohol, methyl alcohol, benzyl alcohol, denatured alcohol or any equivalent thereof known for use in cosmetic products and compositions, which may dry the skin of the user when applied or worn. In certain embodiments, the alcohol may comprise one or more of caprylic alcohol, cetearyl alcohol, cetyl alcohol, decyl alcohol, isostearyl alcohol, lauryl alcohol, myristyl alcohol, oleyl alcohol and stearyl alcohol.

As used herein a wax may refer to a lipophilic compound that is a malleable solid at ambient temperature. Waxes may include mineral waxes, which are those derived from the decomposition of carbon-based organisms, such as petrolatum and paraffin. Additional non-limiting exemplary mineral waxes include cera microcristallina, microcrystalline wax, ozokerite, ceresine isoparaffin, and synthetic wax. It is contemplated that waxes may also comprise natural waxes, such as those derived from plant or animal sources. Exemplary plant derived waxes may include hydrogenated hemp seed oil, hydrogenated soy bean oil, hydrogenated castor oil, hydrogenated olive oil, hydrogenated rapeseed oil, and hydrogenated avocado oil. Exemplary waxes derived from animal sources may include honey, beeswax, lanolin, squalene, carmine, allantoin, ambergris, and placenta. It would be known to the skilled person that certain compounds, such as for example, cetyl alcohol, may be classified as both an alcohol and a wax. It would also be known to the skilled person that wax is often provided in cosmetic compositions to provide waterproofing.

As used herein “a natural component” may comprise any component that is derived from a natural source, such as for example derived from a plant. In certain embodiments, the natural component may be isolated or derived from a natural source. In certain embodiments, the natural component may be a component synthesized to mimic a component present in a natural source. In certain embodiments, the natural component may undergo minimal processing, wherein said minimal processing may comprise hydrolysis. In certain embodiments, the minimal processing may comprise any processing required to prepare the natural component for inclusion into a cosmetic composition or product, such as suspension in a solvent or formation of an emulsion containing the natural component. In certain embodiments, the natural component may be any natural component approved for use in cosmetic products by a regulatory entity, such as, for example, the Food and Drugs Act in the United States of America, Health Canada in Canada, the Industrial Chemicals Act in Australia, the Environmental Protection Authority (Cosmetic Products Group Standard) in New Zealand, the Cosmetic Supervision and Administration Regulation in China, or any equivalent international, national, local or non-governmental regulatory body.

In certain embodiments, the composition or product may comprise or consist of one or more natural component. In certain embodiments, the one or more natural component may comprise or consist of Pyrus malus fruit extract, Glycine soja oil, Butyrospermum parkii butter, Aloe barbadensis leaf juice, Calanthe discolor extract, Laminaria digitata extract, Saccharomyces cerevisiae extract, Camellia sinensis leaf extract, Coffea arabica seedcake extract, Euterpe oleracea fruit extract, Garcinia mangostana fruit extract, Lycium barbarum fruit extract, Morinda citrifolia fruit extract, Orchis mascula flower extract, Punica granatum fruit extract and/or hydrolyzed Opuntia ficus-indica flower extract. The skilled person, in light of the teachings herein, would be able to select an appropriate source for the natural components for their inclusion in the composition or product.

As used herein “a coloring agent” may be any agent included in a cosmetic composition or product that provides a color to the composition or product and/or provides a color to the skin of the user when the product or composition is applied. In certain embodiments, the coloring agent may be any coloring agent approved for use in cosmetic products by a regulatory entity, such as, for example, the Food and Drugs Act in the United States of America, Health Canada in Canada, the Industrial Chemicals Act in Australia, the Environmental Protection Authority (Cosmetic Products Group Standard) in New Zealand, the Cosmetic Supervision and Administration Regulation in China, or any equivalent international, national, local or private regulatory body. In certain embodiments, the one or more coloring agent may comprise one or more pigment. The skilled person, in light of the teachings herein, would be able to select an appropriate source for the coloring agent for their inclusion in the composition or product.

In certain embodiments, the cosmetic composition or product may comprise at least one coloring agent. In certain embodiments, the cosmetic composition or product may comprise at least one coloring agent and the at least one coloring agent may be a plurality of coloring agents, wherein the coloring agents may be combined to obtain a desired color, such as for example, Bella, Nude Pink, Blush, Napa, Praline Rose, Pink Champagne, Blu-Red, Caramel Apple, Beige Champagne, or Sheer Berry. In certain embodiments, the at least one coloring agent may comprise CI 10006, CI 10020, CI 10316, CI 11680, CI 11710, CI 11725, CI 11920, CI 12010, CI 12085, CI 12120, CI 12370, CI 12420, CI 12480, CI 12490, CI 12700, CI 13015, CI 14270, CI 14700, CI 14720, CI 14815, CI 15510, CI 15525, CI 15580, CI 15620, CI 15630, CI 15800, CI 15850, CI 15865, CI 15880, CI 15980, CI 15985, CI 16035, CI 16185, CI 16230, CI 16255, CI 16290, CI 17200, CI 18050, CI 18130, CI 18690, CI 18736, CI 18820, CI 18965, CI 19140, CI 20040, CI 20470, CI 21100, CI 21108, CI 21230, CI 24790, CI 26100, CI 27755, CI 28440, CI 40215, CI 40800, CI 40820, CI 40825, CI 40850, CI 42045, CI 42051, CI 42053, CI 42080, CI 42090, CI 42100, CI 42170, CI 42520, CI 42735, CI 44090, CI 45100, CI 45190, CI 45220, CI 45350, CI 45370, CI 45380, CI 45396, CI 45405, CI 45410, CI 45430, CI 47000, CI 47005, CI 50325, CI 50420, CI 51319, CI 58000, CI 59040, CI 60724, CI 60725, CI 60730, CI 61565, CI 61570, CI 61585, CI 62045, CI 69800, CI 69825, CI 71105, CI 73000, CI 73015, CI 73360, CI 73385, CI 73900, CI 73915, CI 74100, CI 74160, CI 74180, CI 74260, CI 75100, CI 75120, CI 75125, CI 75130, CI 75135, CI 75170, CI 75300, CI 75470, CI 75810, CI 77000, CI 77002, CI 77004, CI 77007, CI 77015, CI 77019, CI 77120, CI 77163, CI 77220, CI 77231, CI 77266, CI 77267, CI 77268:1, CI 77288, CI 77289, CI 77346, CI 77400, CI 77480, CI 77489, CI 77491, CI 77492, CI 77499, CI 77510, CI 77713, CI 77742, CI 77745, CI 77820, CI 77891, CI 77947, Lactoflavin, Caramel, Capsanthin, capsorubin, Beetroot red, Anthocyanins, Aluminum stearate, Zinc stearate, Magnesium stearate, Calcium stearate, Bromothymol blue, Bromocresol green, Acid red 195, any lakes and salts formed by the listed coloring agent, or any combination thereof. In certain embodiments, the at least one color agent may comprise or consist of CI 42090, CI 15850, CI 19140, CI 77491 and CI 77891.

It is contemplated that in certain embodiments, the at least coloring agent may be any combination of coloring agent resulting in the desired color of the composition or product for the user or manufacturer. The skilled person, in light of the teachings herein, would appreciate that it is possible to provide a different color by modulating the combination of the at least one coloring agent, without substantially changing any other properties of the cosmetic as described herein, such as for example length of wear, waterproof, or resistance to transferring. The skilled person, in light of the teachings herein, would be able to provide the at least one coloring agent to provide a cosmetic composition or product as described herein.

In certain embodiments, the composition or product may comprise at least one additional component. The at least one additional component may comprise or consist of cyclopentasiloxane; isododecane; dimethicone crosspolymer; dimethicone; acrylates copolymer; PEG-10 dimethicone; disteardimonium hectorite; glycerin; sodium chloride; phenoxyethanol; polyhydroxystearic acid; silica; glucose; ethylhexylglycerin; aroma and flavor; tocopheryl acetate; panthenol; butylene glycol; urea; glucosamine HCL; tocopherol; citric acid; xanthan gum; gluconolactone; 1,2-hexanediol; caprylyl glycol; sodium benzoate; and calcium gluconate. In certain embodiments, the at least one additional component may be a coloring agent and/or a natural component. The skilled person, in light of the teachings herein, would be able to select an appropriate source for the additional component for their inclusion in the composition or product.

In certain embodiments, it is contemplated that the cosmetic composition or product is substantially resistant to smearing, smudging, and/or transfer from a user's skin, such as the lips. In certain embodiments, it is contemplated that the cosmetic composition or product is completely resistant to smearing, smudging, and/or transfer from a user's skin, such as the lips. A skilled person, in light of the teachings herein would be able to select an appropriate method to determine the resistance to smearing, smudging, and/or transfer, such as, for example, those provided in the Examples.

In certain embodiments, the composition or product may be non-drying when applied to the user's skin. In certain embodiments, the composition or product may be non-drying when applied to the user's lips. Dry skin may be characterized as rough, scaly, and/or flaky skin surface, especially in low humidity conditions and may be associated with the somatory sensations of tightness, itch, and/or pain. As used herein, non-drying may refer to a product or composition that does not dry the skin of the user when worn. It is contemplated that the compositions and products as described herein do not dry the skin after a single use or repeated use by the user. As used herein, “moisturizing” and “moisturizing ability” may refer to the ability of the product or composition to increase or maintain the water content of the skin of the user where the product or composition is applied, such as the lips. As used herein, “conditioning” or “conditioning ability” may refer to the ability of the product or composition to improve the users perceived skin softness and smoothness, which may be as a result of increased moisturizing ability.

In certain embodiments, the composition or product may not cause discomfort when applied to a user's skin. In certain embodiments, the composition or product may not cause discomfort when it is applied to a user's lips. In certain embodiments, the composition or product may not cause discomfort when applied once to a user's lips, or when applied multiple times to a user's lips, such as two or more times. Discomfort as used herein, may refer to any uncomfortable sensation experienced by the user when the product or composition is applied. In certain embodiments, discomfort or an uncomfortable sensation may refer to one or more of: pain, stinging, redness, irritation, burning, or any equivalent uncomfortable sensation known to the skilled person to be associated with cosmetic products or compositions, particularly those to be applied to the lips of a user.

In certain embodiments, the composition or product is for a single use, wherein a single use may refer to a single application of the product or composition on the lips of the user to obtain the desired outcome, such as color or moisture level. In certain embodiments, the composition or product is for multiple use, wherein the user may optionally apply the product or composition two or more times to achieve a desired color and/or moisture level of the skin to which the product or composition is applied, such as the lips of the user. In certain embodiments, the composition or product is not for use prior to the application of a lip gloss. It would be known to the skilled person that lip gloss may be applied subsequently to certain lip cosmetics such as lip balms, lip sticks, and/or lip creams to enhance the appearance or performance of the lip balm, lip stick or lip cream. It may be preferable to a user to provide a product or composition for application to the skin of the user, such as the lips, which does not require subsequent application of lip gloss to achieve the desired outcome, such as color and/or moisture level.

In certain embodiments, the composition or product may be “gluten free”. As used herein, “gluten” refers to a protein found combined with starch in the endosperm of most cereals. Gluten constitutes approximately 80% of the protein contained in wheat and is composed of the proteins gliadine and glutenine. Gluten may be found in most cereals (e.g., wheat, rye and barley) and their end products. No gluten may be found contained in rice, soybean (soya), maize (corn), buckwheat, and sunflower seeds, for example.

In certain embodiments, the product or composition may be substantially free or devoid of animal by-product. As used herein, a composition or product that is “animal by-product free” may be substantially free or devoid of any animal by-product. As used herein, an animal by-product may be any product derived from any organism classified in the “Animal kingdom”, wherein said organisms may be multi-cellular, eukaryotic organisms of the biological kingdom Animalia. In certain embodiments, the product or composition may be certified as vegan or appropriate for use by vegan user's requiring a vegan product. As used herein, a vegan product may be certified as such by an appropriate regulatory body or be known to not contain any components which are not known as vegan. Vegan products may not comprise any product containing a component derived from an organism of the kingdom Animalia.

In certain embodiments as described herein, the product or composition may be substantially free or devoid of parabens. Parabens as used herein may refer to esters of para-hydroxybenzoic acid, such as, but not limited to, methylparaben, ethylparaben, propylparaben, butylparaben, isobutylparaben, isopropylparaben, benzylparaben, heptylparaben, and any sodium salts thereof.

In certain embodiments as described herein, the product or composition may comprise flavor/aroma. In certain embodiments, flavor/aroma may refer to both flavor and aroma, or flavor or aroma. In certain embodiments, the flavor and aroma may be any flavoring and/or aroma agent known to the skilled person to be appropriate for inclusion in a cosmetic composition and product in view of local regulations. In certain embodiments, the flavor/aroma may be any natural flavor/aroma, such as by way of non-limiting example, Vanillin, Ethyl Vanillin, Menthol, Racementhol, Apple Manzanate, Ethyl 2-methylpentanoate, Banana, Isoamyl Acetate, Isopentyl Acetate, Coconut, γ-Octalactone, Cinnamon, Cinnamaldehyde, Grape, Methyl Anthranilate, caraway, Carvone, Limonene, Pear, Ethyl Decadienoate, Fruity, Ethyl Propionate, Truffle, 2,4-Dithiapentane, Wintergreen, Methyl Salicylate, Cherry, Allyl Benzoate, Pineapple, Allyl Hexanoate, Allyl Caproate, Anise, Anethole, Anisaldehyde, Lemon, 3,7-Dimethyl-2,6-Octadienal, Orange, d-Limonene, Ethyl Butyrate, Peppermint, menthyl Acetate, Mango, 3-hydroxy-4,5-dimethyl-2 (5H)-furanone, ethyl-2-methybutanoate, (E,Z)-2,6-nonadienal, 1-(E,Z)-undecatriene, γ-octalactone, ethyl butanoate, Spearmint, Mint, Ketone 1-Carvone, Limonene, Cineole, Ginger, Gingerol, d-Camphene, ß-Phellandrene, Caramel, 3,5-Dimethyl-1,2-Cyclopentadione, Lavender, Linalyl Acetate, Clove, Eugenol, Rose, Geraniol, I-Citronellol, Peach, Aldehyde C-14 Peach, Gamma-Undecanolactone, Raspberry, Raspberry Ketone, 4-(4-Hydroxyphenyl) butan-2-one, Strawberry, Ethyl Methylphenylglycidate, Chocolate Flavor, Cocoa, Cocoa liquor, Cardamon, Limonene, Cineol, Wild cherry, Prunasin, Methionine, Maltol, Ethyl Maltol, Denatonium Benzoate, Ethyl Acetate, Ethyl Lactate, Monosodium Glutamate, Citric Acid Monohydrate, Dibutyl Sebacate, Adipic Acid, Capric acid, Malic Acid, Fumaric Acid, Sodium Metabisulfite, Thymol, Thyme oil, citral, acetoin, Massoia Lactone, any equivalent known to the skilled person or any combination thereof.

In certain embodiments, the cosmetic composition or product may include a combination of one or more structuring agents, emollients, moisturizers, oils, essential oils, lipids, thickening agents, stability agents, shine enhancers, natural flavors, natural aroma, artificial flavors, artificial aroma, odorants, sunscreen agents (for example UV blocking agents), natural components, coloring agents, cosmetic ingredients, additional components and/or antioxidants. The aforementioned groups are not mutually exclusive as, for example, an oil might also be an emollient, a wax may be an alcohol, or a moisturizer may be a natural component. An extensive variation of such ingredients may be used. Examples of some exemplary ingredients and equivalent thereof are provided herein.

In certain embodiments, the composition or product may comprise or consist of components, such as for example, water, isododecane and trisiloxane each at an amount of between about 10% to about 25% including 10%, 10.05%, 10.1%, 10.15%, 10.2%, 10.25%, 10.3%, 10.35%, 10.4%, 10.45%, 10.5%, 10.55%, 10.6%, 10.65%, 10.7%, 10.75%, 10.8%, 10.85%, 10.9%, 10.95%, 11%, 11.05%, 11.1%, 11.15%, 11.2%, 11.25%, 11.3%, 11.35%, 11.4%, 11.45%, 11.5%, 11.55%, 11.6%, 11.65%, 11.7%, 11.75%, 11.8%, 11.85%, 11.9%, 11.95%, 12%, 12.05%, 12.1%, 12.15%, 12.2%, 12.25%, 12.3%, 12.35%, 12.4%, 12.45%, 12.5%, 12.55%, 12.6%, 12.65%, 12.7%, 12.75%, 12.8%, 12.85%, 12.9%, 12.95%, 13%, 13.05%, 13.1%, 13.15%, 13.2%, 13.25%, 13.3%, 13.35%, 13.4%, 13.45%, 13.5%, 13.55%, 13.6%, 13.65%, 13.7%, 13.75%, 13.8%, 13.85%, 13.9%, 13.95%, 14%, 14.05%, 14.1%, 14.15%, 14.2%, 14.25%, 14.3%, 14.35%, 14.4%, 14.45%, 14.5%, 14.55%, 14.6%, 14.65%, 14.7%, 14.75%, 14.8%, 14.85%, 14.9%, 14.95%, 15%, 15.05%, 15.1%, 15.15%, 15.2%, 15.25%, 15.3%, 15.35%, 15.4%, 15.45%, 15.5%, 15.55%, 15.6%, 15.65%, 15.7%, 15.75%, 15.8%, 15.85%, 15.9%, 15.95%, 16%, 16.05%, 16.1%, 16.15%, 16.2%, 16.25%, 16.3%, 16.35%, 16.4%, 16.45%, 16.5%, 16.55%, 16.6%, 16.65%, 16.7%, 16.75%, 16.8%, 16.85%, 16.9%, 16.95%, 17%, 17.05%, 17.1%, 17.15%, 17.2%, 17.25%, 17.3%, 17.35%, 17.4%, 17.45%, 17.5%, 17.55%, 17.6%, 17.65%, 17.7%, 17.75%, 17.8%, 17.85%, 17.9%, 17.95%, 18%, 18.05%, 18.1%, 18.15%, 18.2%, 18.25%, 18.3%, 18.35%, 18.4%, 18.45%, 18.5%, 18.55%, 18.6%, 18.65%, 18.7%, 18.75%, 18.8%, 18.85%, 18.9%, 18.95%, 19%, 19.05%, 19.1%, 19.15%, 19.2%, 19.25%, 19.3%, 19.35%, 19.4%, 19.45%, 19.5%, 19.55%, 19.6%, 19.65%, 19.7%, 19.75%, 19.8%, 19.85%, 19.9%, 19.95%, 20%, 20.05%, 20.1%, 20.15%, 20.2%, 20.25%, 20.3%, 20.35%, 20.4%, 20.45%, 20.5%, 20.55%, 20.6%, 20.65%, 20.7%, 20.75%, 20.8%, 20.85%, 20.9%, 20.95%, 21%, 21.05%, 21.1%, 21.15%, 21.2%, 21.25%, 21.3%, 21.35%, 21.4%, 21.45%, 21.5%, 21.55%, 21.6%, 21.65%, 21.7%, 21.75%, 21.8%, 21.85%, 21.9%, 21.95%, 22%, 22.05%, 22.1%, 22.15%, 22.2%, 22.25%, 22.3%, 22.35%, 22.4%, 22.45%, 22.5%, 22.55%, 22.6%, 22.65%, 22.7%, 22.75%, 22.8%, 22.85%, 22.9%, 22.95%, 23%, 23.05%, 23.1%, 23.15%, 23.2%, 23.25%, 23.3%, 23.35%, 23.4%, 23.45%, 23.5%, 23.55%, 23.6%, 23.65%, 23.7%, 23.75%, 23.8%, 23.85%, 23.9%, 23.95%, 24%, 24.05%, 24.1%, 24.15%, 24.2%, 24.25%, 24.3%, 24.35%, 24.4%, 24.45%, 24.5%, 24.55%, 24.6%, 24.65%, 24.7%, 24.75%, 24.8%, 24.85%, 24.9%, 24.95%, 25%, and any non-integer value not explicitly recited but falling within said range.

In certain embodiments, the composition or product may comprise or consist of components, such as for example, trimethylsiloxysilicate, at an amount of between about 5% to about 10% including 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15%, 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95%, 10%, and any non-integer value not explicitly recited but falling within said range.

In certain embodiments, the composition or product may comprise or consist of components, such as for example, cyclopentasiloxane, dimethicone crosspolymer, hexyl/succinyl dimethicone crosspolymer, dimethicone, acrylates copolymer, CI 77891, PEG-10 dimethicone, CI 77491, disteardimonium hectorite, glycerin, Pyrus malus fruit extract, and xylitylglucoside, at an amount of between about 1% to about 5% including 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5% or any non-integer value not explicitly recited but falling within said range.

In certain embodiments, the composition or product may comprise or consist of components, such as for example, sodium chloride, phenoxyethanol, anhydroxylitol, Glycine soja oil, xylitol, CI 19140, polyhydroxystearic acid, CI 15850, silica and glucose, at an amount of between about 0.1% to about 1% including 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1% or any non-integer value not explicitly recited but falling within said range.

In certain embodiments, the composition or product may comprise or consist of components, such as for example, Butyrospermum parkii butter; ethylhexylglycerin; CI 42090; aroma and flavor; Aloe barbadensis leaf juice; sodium hyaluronate; tocopheryl acetate; panthenol; butylene glycol; Calanthe discolor extract; urea; glucosamine HCL; Laminaria digitata extract; Saccharomyces cerevisiae extract; Camellia sinensis leaf extract; Coffea arabica seedcake extract; Euterpe oleracea fruit extract; Garcinia mangostana fruit extract; Lycium barbarum fruit extract; Morinda citrifolia fruit extract; Orchis mascula flower extract; Punica granatum fruit extract; tocopherol; hydrolyzed Opuntia ficus-indica flower extract; citric acid; xanthan gum; gluconolactone; 1,2-hexanediol; caprylyl glycol; sodium benzoate; and calcium gluconate, at an amount greater than 0% but less than about 0.5% including 0.0005%, 0.001%, 0.0015%, 0.002%, 0.0025%, 0.003%, 0.0035%, 0.004%, 0.0045%, 0.005%, 0.0055%, 0.006%, 0.0065%, 0.007%, 0.0075%, 0.008%, 0.0085%, 0.009%, 0.0095%, 0.01%, 0.0105%, 0.011%, 0.0115%, 0.012%, 0.0125%, 0.013%, 0.0135%, 0.014%, 0.0145%, 0.015%, 0.0155%, 0.016%, 0.0165%, 0.017%, 0.0175%, 0.018%, 0.0185%, 0.019%, 0.0195%, 0.02%, 0.0205%, 0.021%, 0.0215%, 0.022%, 0.0225%, 0.023%, 0.0235%, 0.024%, 0.0245%, 0.025%, 0.0255%, 0.026%, 0.0265%, 0.027%, 0.0275%, 0.028%, 0.0285%, 0.029%, 0.0295%, 0.03%, 0.0305%, 0.031%, 0.0315%, 0.032%, 0.0325%, 0.033%, 0.0335%, 0.034%, 0.0345%, 0.035%, 0.0355%, 0.036%, 0.0365%, 0.037%, 0.0375%, 0.038%, 0.0385%, 0.039%, 0.0395%, 0.04%, 0.0405%, 0.041%, 0.0415%, 0.042%, 0.0425%, 0.043%, 0.0435%, 0.044%, 0.0445%, 0.045%, 0.0455%, 0.046%, 0.0465%, 0.047%, 0.0475%, 0.048%, 0.0485%, 0.049%, 0.0495%, 0.05%, 0.0505%, 0.051%, 0.0515%, 0.052%, 0.0525%, 0.053%, 0.0535%, 0.054%, 0.0545%, 0.055%, 0.0555%, 0.056%, 0.0565%, 0.057%, 0.0575%, 0.058%, 0.0585%, 0.059%, 0.0595%, 0.06%, 0.0605%, 0.061%, 0.0615%, 0.062%, 0.0625%, 0.063%, 0.0635%, 0.064%, 0.0645%, 0.065%, 0.0655%, 0.066%, 0.0665%, 0.067%, 0.0675%, 0.068%, 0.0685%, 0.069%, 0.0695%, 0.07%, 0.0705%, 0.071%, 0.0715%, 0.072%, 0.0725%, 0.073%, 0.0735%, 0.074%, 0.0745%, 0.075%, 0.0755%, 0.076%, 0.0765%, 0.077%, 0.0775%, 0.078%, 0.0785%, 0.079%, 0.0795%, 0.08%, 0.0805%, 0.081%, 0.0815%, 0.082%, 0.0825%, 0.083%, 0.0835%, 0.084%, 0.0845%, 0.085%, 0.0855%, 0.086%, 0.0865%, 0.087%, 0.0875%, 0.088%, 0.0885%, 0.089%, 0.0895%, 0.09%, 0.0905%, 0.091%, 0.0915%, 0.092%, 0.0925%, 0.093%, 0.0935%, 0.094%, 0.0945%, 0.095%, 0.0955%, 0.096%, 0.0965%, 0.097%, 0.0975%, 0.098%, 0.0985%, 0.099%, 0.0995%, 0.1%, 0.1005%, 0.101%, 0.1015%, 0.102%, 0.1025%, 0.103%, 0.1035%, 0.104%, 0.1045%, 0.105%, 0.1055%, 0.106%, 0.1065%, 0.107%, 0.1075%, 0.108%, 0.1085%, 0.109%, 0.1095%, 0.11%, 0.1105%, 0.111%, 0.1115%, 0.112%, 0.1125%, 0.113%, 0.1135%, 0.114%, 0.1145%, 0.115%, 0.1155%, 0.116%, 0.1165%, 0.117%, 0.1175%, 0.118%, 0.1185%, 0.119%, 0.1195%, 0.12%, 0.1205%, 0.121%, 0.1215%, 0.122%, 0.1225%, 0.123%, 0.1235%, 0.124%, 0.1245%, 0.125%, 0.1255%, 0.126%, 0.1265%, 0.127%, 0.1275%, 0.128%, 0.1285%, 0.129%, 0.1295%, 0.13%, 0.1305%, 0.131%, 0.1315%, 0.132%, 0.1325%, 0.133%, 0.1335%, 0.134%, 0.1345%, 0.135%, 0.1355%, 0.136%, 0.1365%, 0.137%, 0.1375%, 0.138%, 0.1385%, 0.139%, 0.1395%, 0.14%, 0.1405%, 0.141%, 0.1415%, 0.142%, 0.1425%, 0.143%, 0.1435%, 0.144%, 0.1445%, 0.145%, 0.1455%, 0.146%, 0.1465%, 0.147%, 0.1475%, 0.148%, 0.1485%, 0.149%, 0.1495%, 0.15%, 0.1505%, 0.151%, 0.1515%, 0.152%, 0.1525%, 0.153%, 0.1535%, 0.154%, 0.1545%, 0.155%, 0.1555%, 0.156%, 0.1565%, 0.157%, 0.1575%, 0.158%, 0.1585%, 0.159%, 0.1595%, 0.16%, 0.1605%, 0.161%, 0.1615%, 0.162%, 0.1625%, 0.163%, 0.1635%, 0.164%, 0.1645%, 0.165%, 0.1655%, 0.166%, 0.1665%, 0.167%, 0.1675%, 0.168%, 0.1685%, 0.169%, 0.1695%, 0.17%, 0.1705%, 0.171%, 0.1715%, 0.172%, 0.1725%, 0.173%, 0.1735%, 0.174%, 0.1745%, 0.175%, 0.1755%, 0.176%, 0.1765%, 0.177%, 0.1775%, 0.178%, 0.1785%, 0.179%, 0.1795%, 0.18%, 0.1805%, 0.181%, 0.1815%, 0.182%, 0.1825%, 0.183%, 0.1835%, 0.184%, 0.1845%, 0.185%, 0.1855%, 0.186%, 0.1865%, 0.187%, 0.1875%, 0.188%, 0.1885%, 0.189%, 0.1895%, 0.19%, 0.1905%, 0.191%, 0.1915%, 0.192%, 0.1925%, 0.193%, 0.1935%, 0.194%, 0.1945%, 0.195%, 0.1955%, 0.196%, 0.1965%, 0.197%, 0.1975%, 0.198%, 0.1985%, 0.199%, 0.1995%, 0.2%, 0.2005%, 0.201%, 0.2015%, 0.202%, 0.2025%, 0.203%, 0.2035%, 0.204%, 0.2045%, 0.205%, 0.2055%, 0.206%, 0.2065%, 0.207%, 0.2075%, 0.208%, 0.2085%, 0.209%, 0.2095%, 0.21%, 0.2105%, 0.211%, 0.2115%, 0.212%, 0.2125%, 0.213%, 0.2135%, 0.214%, 0.2145%, 0.215%, 0.2155%, 0.216%, 0.2165%, 0.217%, 0.2175%, 0.218%, 0.2185%, 0.219%, 0.2195%, 0.22%, 0.2205%, 0.221%, 0.2215%, 0.222%, 0.2225%, 0.223%, 0.2235%, 0.224%, 0.2245%, 0.225%, 0.2255%, 0.226%, 0.2265%, 0.227%, 0.2275%, 0.228%, 0.2285%, 0.229%, 0.2295%, 0.23%, 0.2305%, 0.231%, 0.2315%, 0.232%, 0.2325%, 0.233%, 0.2335%, 0.234%, 0.2345%, 0.235%, 0.2355%, 0.236%, 0.2365%, 0.237%, 0.2375%, 0.238%, 0.2385%, 0.239%, 0.2395%, 0.24%, 0.2405%, 0.241%, 0.2415%, 0.242%, 0.2425%, 0.243%, 0.2435%, 0.244%, 0.2445%, 0.245%, 0.2455%, 0.246%, 0.2465%, 0.247%, 0.2475%, 0.248%, 0.2485%, 0.249%, 0.2495%, 0.25%, 0.2505%, 0.251%, 0.2515%, 0.252%, 0.2525%, 0.253%, 0.2535%, 0.254%, 0.2545%, 0.255%, 0.2555%, 0.256%, 0.2565%, 0.257%, 0.2575%, 0.258%, 0.2585%, 0.259%, 0.2595%, 0.26%, 0.2605%, 0.261%, 0.2615%, 0.262%, 0.2625%, 0.263%, 0.2635%, 0.264%, 0.2645%, 0.265%, 0.2655%, 0.266%, 0.2665%, 0.267%, 0.2675%, 0.268%, 0.2685%, 0.269%, 0.2695%, 0.27%, 0.2705%, 0.271%, 0.2715%, 0.272%, 0.2725%, 0.273%, 0.2735%, 0.274%, 0.2745%, 0.275%, 0.2755%, 0.276%, 0.2765%, 0.277%, 0.2775%, 0.278%, 0.2785%, 0.279%, 0.2795%, 0.28%, 0.2805%, 0.281%, 0.2815%, 0.282%, 0.2825%, 0.283%, 0.2835%, 0.284%, 0.2845%, 0.285%, 0.2855%, 0.286%, 0.2865%, 0.287%, 0.2875%, 0.288%, 0.2885%, 0.289%, 0.2895%, 0.29%, 0.2905%, 0.291%, 0.2915%, 0.292%, 0.2925%, 0.293%, 0.2935%, 0.294%, 0.2945%, 0.295%, 0.2955%, 0.296%, 0.2965%, 0.297%, 0.2975%, 0.298%, 0.2985%, 0.299%, 0.2995%, 0.3%, 0.3005%, 0.301%, 0.3015%, 0.302%, 0.3025%, 0.303%, 0.3035%, 0.304%, 0.3045%, 0.305%, 0.3055%, 0.306%, 0.3065%, 0.307%, 0.3075%, 0.308%, 0.3085%, 0.309%, 0.3095%, 0.31%, 0.3105%, 0.311%, 0.3115%, 0.312%, 0.3125%, 0.313%, 0.3135%, 0.314%, 0.3145%, 0.315%, 0.3155%, 0.316%, 0.3165%, 0.317%, 0.3175%, 0.318%, 0.3185%, 0.319%, 0.3195%, 0.32%, 0.3205%, 0.321%, 0.3215%, 0.322%, 0.3225%, 0.323%, 0.3235%, 0.324%, 0.3245%, 0.325%, 0.3255%, 0.326%, 0.3265%, 0.327%, 0.3275%, 0.328%, 0.3285%, 0.329%, 0.3295%, 0.33%, 0.3305%, 0.331%, 0.3315%, 0.332%, 0.3325%, 0.333%, 0.3335%, 0.334%, 0.3345%, 0.335%, 0.3355%, 0.336%, 0.3365%, 0.337%, 0.3375%, 0.338%, 0.3385%, 0.339%, 0.3395%, 0.34%, 0.3405%, 0.341%, 0.3415%, 0.342%, 0.3425%, 0.343%, 0.3435%, 0.344%, 0.3445%, 0.345%, 0.3455%, 0.346%, 0.3465%, 0.347%, 0.3475%, 0.348%, 0.3485%, 0.349%, 0.3495%, 0.35%, 0.3505%, 0.351%, 0.3515%, 0.352%, 0.3525%, 0.353%, 0.3535%, 0.354%, 0.3545%, 0.355%, 0.3555%, 0.356%, 0.3565%, 0.357%, 0.3575%, 0.358%, 0.3585%, 0.359%, 0.3595%, 0.36%, 0.3605%, 0.361%, 0.3615%, 0.362%, 0.3625%, 0.363%, 0.3635%, 0.364%, 0.3645%, 0.365%, 0.3655%, 0.366%, 0.3665%, 0.367%, 0.3675%, 0.368%, 0.3685%, 0.369%, 0.3695%, 0.37%, 0.3705%, 0.371%, 0.3715%, 0.372%, 0.3725%, 0.373%, 0.3735%, 0.374%, 0.3745%, 0.375%, 0.3755%, 0.376%, 0.3765%, 0.377%, 0.3775%, 0.378%, 0.3785%, 0.379%, 0.3795%, 0.38%, 0.3805%, 0.381%, 0.3815%, 0.382%, 0.3825%, 0.383%, 0.3835%, 0.384%, 0.3845%, 0.385%, 0.3855%, 0.386%, 0.3865%, 0.387%, 0.3875%, 0.388%, 0.3885%, 0.389%, 0.3895%, 0.39%, 0.3905%, 0.391%, 0.3915%, 0.392%, 0.3925%, 0.393%, 0.3935%, 0.394%, 0.3945%, 0.395%, 0.3955%, 0.396%, 0.3965%, 0.397%, 0.3975%, 0.398%, 0.3985%, 0.399%, 0.3995%, 0.4%, 0.4005%, 0.401%, 0.4015%, 0.402%, 0.4025%, 0.403%, 0.4035%, 0.404%, 0.4045%, 0.405%, 0.4055%, 0.406%, 0.4065%, 0.407%, 0.4075%, 0.408%, 0.4085%, 0.409%, 0.4095%, 0.41%, 0.4105%, 0.411%, 0.4115%, 0.412%, 0.4125%, 0.413%, 0.4135%, 0.414%, 0.4145%, 0.415%, 0.4155%, 0.416%, 0.4165%, 0.417%, 0.4175%, 0.418%, 0.4185%, 0.419%, 0.4195%, 0.42%, 0.4205%, 0.421%, 0.4215%, 0.422%, 0.4225%, 0.423%, 0.4235%, 0.424%, 0.4245%, 0.425%, 0.4255%, 0.426%, 0.4265%, 0.427%, 0.4275%, 0.428%, 0.4285%, 0.429%, 0.4295%, 0.43%, 0.4305%, 0.431%, 0.4315%, 0.432%, 0.4325%, 0.433%, 0.4335%, 0.434%, 0.4345%, 0.435%, 0.4355%, 0.436%, 0.4365%, 0.437%, 0.4375%, 0.438%, 0.4385%, 0.439%, 0.4395%, 0.44%, 0.4405%, 0.441%, 0.4415%, 0.442%, 0.4425%, 0.443%, 0.4435%, 0.444%, 0.4445%, 0.445%, 0.4455%, 0.446%, 0.4465%, 0.447%, 0.4475%, 0.448%, 0.4485%, 0.449%, 0.4495%, 0.45%, 0.4505%, 0.451%, 0.4515%, 0.452%, 0.4525%, 0.453%, 0.4535%, 0.454%, 0.4545%, 0.455%, 0.4555%, 0.456%, 0.4565%, 0.457%, 0.4575%, 0.458%, 0.4585%, 0.459%, 0.4595%, 0.46%, 0.4605%, 0.461%, 0.4615%, 0.462%, 0.4625%, 0.463%, 0.4635%, 0.464%, 0.4645%, 0.465%, 0.4655%, 0.466%, 0.4665%, 0.467%, 0.4675%, 0.468%, 0.4685%, 0.469%, 0.4695%, 0.47%, 0.4705%, 0.471%, 0.4715%, 0.472%, 0.4725%, 0.473%, 0.4735%, 0.474%, 0.4745%, 0.475%, 0.4755%, 0.476%, 0.4765%, 0.477%, 0.4775%, 0.478%, 0.4785%, 0.479%, 0.4795%, 0.48%, 0.4805%, 0.481%, 0.4815%, 0.482%, 0.4825%, 0.483%, 0.4835%, 0.484%, 0.4845%, 0.485%, 0.4855%, 0.486%, 0.4865%, 0.487%, 0.4875%, 0.488%, 0.4885%, 0.489%, 0.4895%, 0.49%, 0.4905%, 0.491%, 0.4915%, 0.492%, 0.4925%, 0.493%, 0.4935%, 0.494%, 0.4945%, 0.495%, 0.4955%, 0.496%, 0.4965%, 0.497%, 0.4975%, 0.498%, 0.4985%, 0.499%, 0.4995%, 0.5% or any non-integer value not explicitly recited but falling within said range.

In certain embodiments, the composition or product may be long-lasting on the user's lips. As used herein long-lasting may refer to a length of time in which the composition or product maintains a desirable appearance on a user once applied to the users lips, wherein long-lasting may be a duration determined by the user. In certain embodiments, long-lasting may refer a length of time as provided in the Examples herein. In certain embodiments, long-lasting may comprise an excellent appearance; a good appearance, or a fair appearance after at least 4 hours, 6 hours, and/or 8 hours following application of the product or composition to the users skin. In certain embodiments, long-lasting may comprise a fair appearance or superior score as determined by the user 6 hours following application of the product or composition to the users skin.

In certain embodiments, the composition or product may be resistant to transfer from the user's lips. As used herein resistant to transfer from the user's lips may refer to an ability to resist transfer from the users lips to other objects. In certain embodiments, resistant to transfer from the user's lips may refer resistance to transfer from the user's lips as provided in the Examples herein. In certain embodiments, resistance to transfer of a product or composition may be determined approximately 1, 2, 3, 4, 5, 6, 7, 8, 9, or 10 minutes after application of the product or composition to the users skin by rubbing the users skin in which the product or composition was applied with a white soft material, such as a towel, and scoring the appearance of the product or composition transference (if any) on the white material according to a scale, such as, 0=No evidence of any transference; 1-3=Slight transference visible; 4-6=Moderate transference visible; and 7-9=Severe transference visible. In certain embodiments, a mean score of in or near the “slight transference visible” category (“0-3”-0 to 3.0) may support a claim of resistant to transfer.

In certain embodiments, the composition or product may be waterproof on the user's lips. As used herein waterproof on the user's lips may refer to an ability to resist loss of the composition or product from the users lips when water contacts the users lips. In certain embodiments, waterproof on the user's lips may refer to waterproof on the user's lips as provided in the Examples herein. In certain embodiments, waterproof of a user's lips may refer to an evaluation of the appearance of the composition or product on the lips or skin of a user following splashing or submerging the lips or skin in water. In certain embodiments, the waterproof ability of a composition or product may be evaluated for the lips and/or skin according to a scale, such as, 4=Excellent appearance; 3=Good appearance; 2=Fair appearance; 1=Poor appearance; and 0=No product visible after splashing or submerging the lips or skin in water. In certain embodiments, a mean score of in or near the “good appearance” category or above (“3”-2.5 to 4.0) may support a claim of a water-proof product or composition.

In certain embodiments as described herein, a percentage of a component in a composition may refer to a weight/weight, a weight/volume, a volume/volume, or a volume/weight percentage of said component in the total composition or product.

### EXAMPLES

The following examples are provided for illustrative purposes and are intended for the person of skill in the art. These examples are provided to demonstrate certain embodiments as described herein, and should not be seen as limiting in any way.

### Example 1—Panel Testing

Cosmetic products for application to the lips containing compositions CK060921A2, CK082421C, and CK061522E1, as well as a reference composition comprising alcohol were submitted for panel testing.

The reference composition comprising Alcohol Denat., Silica, Acrylates/Octylacrylamide Copolymer, Isostearyl Alcohol, Mica, Parfum/Fragrance, Aqua/Water/Eau, Isodonis japonicus Leaf/Stalk Extract, Hypericum perforatum (St. John's Wort) Extract, Paeonia suffruticosa (Tree Peony) Root Extract, Tilia cordata (Linden) Flower Extract, Hydroxypropylcellulose, Butylene Glycol, Benzoic Acid, Citronellol, Benzyl Benzoate, Limonene, and Iron Oxides (CI 77491).

The panel participants were asked to provide their rating from 1-5 for each statement provided in Table 1, wherein a score of 5 reflects the panelist completely agrees and a score of 0 reflects the panelist completely disagrees with the following statements:

TABLE 1
Statements provided for panelists to respond
thereto following application of cosmetic products
Statement

The product goes on very smoothly.
I like the scent.
I like the texture.
The product dries in an acceptable amount of time.
The produce has good color-payoff and coverage with 1 layer.
I like the soft matte finish.
While wearing the product, lips feel conditioned and not dry
The product lasts a good amount of time throughout the day.
I did not experience any uncomfortable sensation, such
as stinging, while wearing this product.
I would incorporate this product formula into my daily
or occasional makeup routine.

Total average scores reflecting the average of the values for each statement for each panelist, as well as the average score for each individual statement across all panelists were both calculated. For example, a panelist providing 5 responses of 3/5 and 5 response of 4/5 would result in an average score for the product for said panelist of 3.5/5. Furthermore, if for a given product, 5 panelists provide a rating of 4/5 and 5 panelists provide a rating of 2/5 for “The product lasts a good amount of time throughout the day”, the average score for “The product lasts a good amount of time throughout the day” for that product would be 3/5.

Panelists were provided with CK060921A2, CK082421C, and CK061522E1, and asked to provide scores based on their experience using the product for each of the statements outlined in Table 1. For reference, panelists were provided with a conventional lip cream composition comprising simple alcohol.

Panelists provided an overall average score of 4.4 for CK061522E1, whereas the reference, CK060921A2 and CK082421C received overall averages scores of 4.2, 3.8 and 3.24, respectively, in response to the statements outlined in Table 1.

Regarding CK060921A2 panelists provided the following exemplary comments in addition to the scores for the statements provided in Table 1. Panelists noted the product transfers with eating and drinking too much, the product is not long lasting on lips enough, and the product took too long to dry/set, with an average of over 2 minutes between layers. Panelists specifically noted:
“I like this formula for the way it goes on and feels. But it transfers a lot. Almost like regular lipstick. It is not a replacement for me for my current alcohol-containing product.”; and
“Long time between application layering. when applying the second or third layer, the previous application would start to come off. This formula felt velvety and dried more like a lipstick. This product did not stay in place. Right away product started coming off.

Regarding CK082421C panelists provided the following exemplary comments in response to the scores outlined for the statement provided in Table 1. Only 4 out of 10 panelists were able to wear the formula without issue. Panelists noted wear time between 2-8 hours, lasting longer than a traditional bullet style lipstick, but less than the reference. The statement of “While wearing the product, lips feel conditioned and not dry” received a score of 2.88.

Regarding, CK061522E1 panelists provided the following exemplary comments in response to the scores outlined for the statement provided in Table 1. 8 out of 10 panelists noted a wear time between 4 and 8 hours and 8 out of 10 panelists were able to wear the formula without discomfort. Panelists specifically noted:
“It's so easy to apply, is very pleasantly scented, has zero sting, and dries down to a gorgeous matte finish”; and
“For people with dry lips like me, it is a really good long lasting lip color that doesn't dry out the lips.”.

### Example 2—Exemplary Products and Compositions

In response to panel testing the composition of CK060921A2 was modified to increase the amount of hexyl/succinyl dimethicone crosspolymer, and the amount of trisiloxane, and to decrease the amount of coloring agent, resulting in the composition of CK082421C.

In response to panel testing the composition of CK082421C was modified to further decrease the amount of coloring agent, and to increase the amounts of xylitol, androxylitol, xylityl glucosides, resulting in the composition of CK061522E1 as outlined below in Table 2.

TABLE 2
An exemplary cosmetic composition CK061522E1 as described herein:

| INCI Ingredient | % Range (w/w) |
|---|---|
| Aqua/Water/Eau | 10%-25% |
| Isododecane | 10%-25% |
| Trisiloxane | 10%-25% |
| Trimethylsiloxysilicate | 5%-10% |
| Cyclopentasiloxane | 1%-5% |
| Dimethicone Crosspolymer | 1%-5% |
| Hexyl/Succinyl Dimethicone Crosspolymer | 1%-5% |
| Dimethicone | 1%-5% |
| Acrylates Copolymer | 1%-5% |
| Titanium Dioxide (CI 77891) | 1%-5% |
| PEG-10 Dimethicone | 1%-5% |
| Iron Oxides (CI 77491) | 1%-5% |
| Disteardimonium Hectorite | 1%-5% |
| Glycerin | 1%-5% |
| Pyrus Malus (Apple) Fruit Extract | 1%-5% |
| Xylitylglucoside | 1%-5% |
| Sodium Chloride | 0.1%-1% |
| Phenoxyethanol | 0.1%-1% |
| Anhydroxylitol | 0.1%-1% |
| Glycine Soja Oil | 0.1%-1% |
| Xylitol | 0.1%-1% |
| Yellow 5 Lake (CI 19140) | 0.1%-1% |
| Polyhydroxystearic Acid | 0.1%-1% |
| Red 7 Lake (CI 15850) | 0.1%-1% |
| Silica | 0.1%-1% |
| Glucose | 0.1%-1% |
| Butyrospermum Parkii (Shea) Butter | 0%>-≤0.5% |
| Ethylhexylglycerin | 0%>-≤0.5% |
| CI 42090 | 0%>-≤0.5% |
| Aroma/Flavor | 0%>-≤0.5% |
| Aloe Barbadensis (Aloe Vera) Leaf Juice | 0%>-≤0.5% |
| Sodium Hyaluronate | 0%>-≤0.5% |
| Tocopheryl Acetate | 0%>-≤0.5% |
| Panthenol | 0%>-≤0.5% |
| Butylene Glycol | 0%>-≤0.5% |
| Calanthe Discolor (Ebine) Extract | 0%>-≤0.5% |
| Urea | 0%>-≤0.5% |
| Glucosamine HCL | 0%>-≤0.5% |
| Laminaria Digitata (Kelp) Extract | 0%>-≤0.5% |
| Saccharomyces Cerevisiae (Yeast) Extract | 0%>-≤0.5% |
| Camellia Sinensis (Green Tea) Leaf Extract | 0%>-≤0.5% |
| Coffea Arabica Seedcake Extract | 0%>-≤0.5% |
| Euterpe Oleracea (Acai) Fruit Extract | 0%>-≤0.5% |
| Garcinia Mangostana (Mangosteen) Fruit Extract | 0%>-≤0.5% |
| Lycium Barbarum (Goji) Fruit Extract | 0%>-≤0.5% |
| Morinda Citrifolia (Noni) Fruit Extract | 0%>-≤0.5% |
| Orchis Mascula (Orchid) Flower Extract | 0%>-≤0.5% |
| Punica Granatum (Pomegranate) Fruit Extract | 0%>-≤0.5% |
| Tocopherol | 0%>-≤0.5% |
| Hydrolyzed Opuntia Ficus-Indica (Prickly Pear) Flower Extract | 0%>-≤0.5% |
| Citric Acid | 0%>-≤0.5% |
| Xanthan Gum | 0%>-≤0.5% |
| Gluconolactone | 0%>-≤0.5% |
| 1,2-Hexanediol | 0%>-≤0.5% |
| Caprylyl Glycol | 0%>-≤0.5% |
| Sodium Benzoate | 0%>-≤0.5% |
| Calcium Gluconate | 0%>-≤0.5% |

### Example 3—Clinical Evaluation

A composition as described herein was provided for panel testing, wherein a total of 32 female subjects between the ages of 19 and 50 years of age were enrolled.

### Example 4—Clinical Evaluation of Length of Wear

Immediately following application of the composition and 4, 6, and 8 following application of the composition, a trained technician and the subjects evaluated the appearance of the lip product on the lips. The technician and subjects were asked to score the appearance of the product according to the following scale for test site appearance: 4=Excellent appearance; 3=Good appearance; 2=Fair appearance; 1=Poor appearance; and 0=No product visible.

TABLE 3
Mean appearance scores for long wear evaluation

Mean Score

| | Technician | Subjects |
|---|---|---|
| Baseline | 4.0 | 4.0 |
| 4 hours | 3.2 | 3.4 |
| 6 hours | 1.9 | 2.3 |
| 8 hours | 0.8 | 1.0 |

Mean scores at baseline (immediately post-application) were compared with those at 4, 6, and 8 hours after application, and there was a decrease in the appearance of the lip product observed by the technician and subjects at all-time points. However, mean scores were in the “good appearance” category (“3”) at 4 hours, in or near the “fair appearance” category (“2”) at 6 hours, and in or near the “poor appearance” category (“1”) at 8 hours for both the technician and subject evaluations.

### Example 5—Clinical Evaluation of Transfer

A trained technician marked one 2 cm×2 cm test site on the forearm of each subject, wherein the forearm was free of any lotions or creams, and then applied the test product to the subject's forearm until an excellent appearance was achieved. In order to determine the “transfer-proof” potential of the test product, approximately 5 minutes after application, the technician gently rubbed the subject's forearm with a white towel and scored the appearance of product transference (if any) on the towel according to the following scale: 0=No evidence of any transference; 1-3=Slight transference visible; 4-6=Moderate transference visible; and 7-9=Severe transference visible. If the mean score was in or near the “slight transference visible” category (“0-3”-0 to 3.0) a transfer-proof claim was considered to be supported.

TABLE 4
Mean appearance scores for transfer proof evaluation

| | Mean score |
|---|---|
| After rubbing | 0.4 |

There was slight transference of the test product observed by the technician. The mean score remained near the “no evidence of any transference” category (“0”) after rubbing.

### Example 6—Clinical Evaluation of Waterproof

A trained technician marked one 2 cm×2 cm test site on the subject's forearm then applied the test product to the subject's forearm until an excellent appearance was achieved. Immediately after application, the technician and subject evaluated the appearance of the lip product on the forearm. Five minutes after application to the forearm, the subject submerged their forearm under water (27° C.) for approximately 30 seconds and the evaluations were repeated.

Under the supervision of the trained technician, subjects applied the lipstick to their lips according to the Sponsor's use instructions until an appearance score of “4” was achieved. Immediately after application, the technician and subject evaluated the appearance of the lipstick on the lips. Five minutes after application to the lips, subjects splashed water on their lips for approximately 30 seconds. The technician and subject then scored the appearance of the lipstick.

Evaluation for the waterproof capabilities of the product were evaluated for the lips and forearm according to the following scale: 4=Excellent appearance; 3=Good appearance; 2=Fair appearance; 1=Poor appearance; and 0=No product visible.

Evaluations were based on a comparison of baseline versus after water exposure. If the mean score was in or near the “good appearance” category or above (“3”-2.5 to 4.0) a water-proof claim was considered to be supported.

TABLE 5
Mean appearance scores for waterproof capabilities on the lips

| | Technician Evaluation | | Subject Evaluation | |
|---|---|---|---|---|
| | | After water | | After water |
| | Baseline | exposure | Baseline | exposure |
| Mean Score | 4.0 | 4.0 | 4.0 | 4.0 |

When compared with baseline (before splashing), there was no change in the appearance of the product observed by either the trained technician or subjects (mean score of 4.0 vs 4.0) after the lips were splashed with water. The mean scores remained in the “excellent appearance” category (“4”) for both the trained technician and subject evaluations.

TABLE 6
Mean appearance scores for waterproof capabilities on the forearm

| | Technician Evaluation | | Subject Evaluation | |
|---|---|---|---|---|
| | | After water | | After water |
| | Baseline | exposure | Baseline | exposure |
| Mean Score | 4.0 | 4.0 | 4.0 | 4.0 |

When compared with baseline (before submersion), there was no change in the appearance of the test product observed by either the trained technician or subjects (mean score of 4.0 vs. 4.0) after the forearm was submerged in water for 30 seconds. Both mean scores remained in the “excellent appearance” category (“4”) for both the trained technician and subject evaluations.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

As used herein, the term “substantially” refers to an approximately +/−5% variation from a given value. If a value is not used, then substantially means almost completely, but perhaps with some variation, contamination and/or additional component. In some embodiments, “substantially” may include completely.

It should be understood that the compositions, products, uses and methods are described in terms of “comprising,” “containing,” or “including” various components or steps, the compositions, products, uses and methods can also “consist essentially of” or “consist of the various components and steps”. Moreover, the indefinite articles “a” or “an”, as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b” or, “from a to b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be referenced herein, the definitions that are consistent with this specification should be adopted.

Many obvious variations of the embodiments set out herein will suggest themselves to those skilled in the art in light of the present disclosure. Such obvious variations are within the full intended scope of the appended claims.

What is claimed is:

1. A cosmetic composition comprising water, trisiloxane, trimethylsiloxysilicate, sodium hyaluronate, xylitol, androxylitol, xylityl glucosides, hexyl/succinyl dimethicone crosspolymer, at least one natural component, at least one coloring agent, and at least one additional component.

2. The cosmetic composition of claim 1, wherein the at least one natural component, the at least one coloring agent and the at least one additional component do not comprise a simple alcohol, a wax, or both.

3. The cosmetic composition of claim 2, wherein the wax is a mineral wax, a wax derived from an animal product, or a non-vegan wax.

4. The composition of claim 2 or 3, wherein the simple alcohol is ethyl alcohol, isopropyl alcohol, methyl alcohol, benzyl alcohol, denatured alcohol, or any combination thereof.

5. The cosmetic composition of any one of claims 1-4, wherein the at least one natural component comprises Pyrus malus fruit extract, Glycine soja oil, Butyrospermum parkii butter, Aloe barbadensis leaf juice, Calanthe discolor extract, Laminaria digitata extract, Saccharomyces cerevisiae extract, Camellia sinensis leaf extract, Coffea arabica seedcake extract, Euterpe oleracea fruit extract, Garcinia mangostana fruit extract, Lycium barbarum fruit extract, Morinda citrifolia fruit extract, Orchis mascula flower extract, Punica granatum fruit extract and hydrolyzed Opuntia ficus-indica flower extract.

6. The cosmetic composition of any one of claims 1-5, wherein the at least one coloring agent comprises at least one white coloring agent, at least one red coloring agent, at least one blue coloring agent, at least one saffron coloring agent and at least one yellow coloring agent.

7. The composition of any one of claims 1-6, wherein the at least one coloring agent comprises: CI 10006, CI 10020, CI 10316, CI 11680, CI 11710, CI 11725, CI 11920, CI 12010, CI 12085, CI 12120, CI 12370, CI 12420, CI 12480, CI 12490, CI 12700, CI 13015, CI 14270, CI 14700, CI 14720, CI 14815, CI 15510, CI 15525, CI 15580, CI 15620, CI 15630, CI 15800, CI 15850, CI 15865, CI 15880, CI 15980, CI 15985, CI 16035, CI 16185, CI 16230, CI 16255, CI 16290, CI 17200, CI 18050, CI 18130, CI 18690, CI 18736, CI 18820, CI 18965, CI 19140, CI 20040, CI 20470, CI 21100, CI 21108, CI 21230, CI 24790, CI 26100, CI 27755, CI 28440, CI 40215, CI 40800, CI 40820, CI 40825, CI 40850, CI 42045, CI 42051, CI 42053, CI 42080, CI 42090, CI 42100, CI 42170, CI 42520, CI 42735, CI 44090, CI 45100, CI 45190, CI 45220, CI 45350, CI 45370, CI 45380, CI 45396, CI 45405, CI 45410, CI 45430, CI 47000, CI 47005, CI 50325, CI 50420, CI 51319, CI 58000, CI 59040, CI 60724, CI 60725, CI 60730, CI 61565, CI 61570, CI 61585, CI 62045, CI 69800, CI 69825, CI 71105, CI 73000, CI 73015, CI 73360, CI 73385, CI 73900, CI 73915, CI 74100, CI 74160, CI 74180, CI 74260, CI 75100, CI 75120, CI 75125, CI 75130, CI 75135, CI 75170, CI 75300, CI 75470, CI 75810, CI 77000, CI 77002, CI 77004, CI 77007, CI 77015, CI 77019, CI 77120, CI 77163, CI 77220, CI 77231, CI 77266, CI 77267, CI 77268:1, CI 77288, CI 77289, CI 77346, CI 77400, CI 77480, CI 77489, CI 77491, CI 77492, CI 77499, CI 77510, CI 77713, CI 77742, CI 77745, CI 77820, CI 77891, CI 77947, Lactoflavin, Caramel, Capsanthin, capsorubin, Beetroot red, Anthocyanins, Aluminum stearate, Zinc stearate, Magnesium stearate, Calcium stearate, Bromothymol blue, Bromocresol green, Acid red 195, any lakes and salts formed by the listed coloring agent, or any combination thereof.

8. The cosmetic composition of any one of claims 1-7, wherein the at least one coloring agent comprises CI 42090, CI 15850, CI 19140, CI 77491 and CI 77891.

9. The cosmetic composition of any one of claims 1-8, wherein the at least one additional component comprises: cyclopentasiloxane; isododecane; dimethicone crosspolymer; dimethicone; acrylates copolymer; PEG-10 dimethicone; disteardimonium hectorite; glycerin; sodium chloride; phenoxyethanol; polyhydroxystearic acid; silica; glucose; ethylhexylglycerin; aroma and flavor; tocopheryl acetate; panthenol; butylene glycol; urea; glucosamine HCL; tocopherol; citric acid; xanthan gum; gluconolactone; 1,2-hexanediol; caprylyl glycol; sodium benzoate; and calcium gluconate.

10. A cosmetic composition comprising: water, isododecane, trisiloxane, trimethylsiloxysilicate, cyclopentasiloxane, dimethicone crosspolymer, hexyl/succinyl dimethicone crosspolymer, dimethicone, acrylates copolymer, PEG-10 dimethicone, disteardimonium hectorite, glycerin, Pyrus malus fruit extract, xylitylglucoside, sodium chloride, phenoxyethanol, anhydroxylitol, Glycine soja oil, xylitol, polyhydroxystearic acid, silica, glucose, Butyrospermum parkii butter, ethylhexylglycerin, aroma and flavor, Aloe barbadensis leaf juice, sodium hyaluronate, tocopheryl acetate, panthenol, butylene glycol, Calanthe discolor extract, urea, glucosamine HCl, Laminaria digitata extract, Saccharomyces cerevisiae extract, Camellia sinensis leaf extract, Coffea arabica seedcake extract, Euterpe oleracea fruit extract, Garcinia mangostana fruit extract, Lycium barbarum fruit extract, Morinda citrifolia fruit extract, Orchis mascula flower extract, Punica granatum fruit extract, tocopherol, hydrolyzed Opuntia ficus-indica flower extract, citric acid, xanthan gum, gluconolactone, 1,2-hexanediol, caprylyl glycol, sodium benzoate, calcium gluconate, and at least one coloring agent.

11. A cosmetic composition comprising: water, isododecane, trisiloxane, trimethylsiloxysilicate, cyclopentasiloxane, dimethicone crosspolymer, hexyl/succinyl dimethicone crosspolymer, dimethicone, acrylates copolymer, CI 77891, PEG-10 dimethicone, CI 77491, disteardimonium hectorite, glycerin, Pyrus malus fruit extract, xylitylglucoside, sodium chloride, phenoxyethanol, anhydroxylitol, Glycine soja oil, xylitol, CI 19140, polyhydroxystearic acid, CI 15850, silica, glucose, Butyrospermum parkii butter, ethylhexylglycerin, CI 42090, aroma and flavor, Aloe barbadensis leaf juice, sodium hyaluronate, tocopheryl acetate, panthenol, butylene glycol, Calanthe discolor extract, urea, glucosamine HCl, Laminaria digitata extract, Saccharomyces cerevisiae extract, Camellia sinensis leaf extract, Coffea arabica seedcake extract, Euterpe oleracea fruit extract, Garcinia mangostana fruit extract, Lycium barbarum fruit extract, Morinda citrifolia fruit extract, Orchis mascula flower extract, Punica granatum fruit extract, tocopherol, hydrolyzed Opuntia ficus-indica flower extract, citric acid, xanthan gum, gluconolactone, 1,2-hexanediol, caprylyl glycol, sodium benzoate, and calcium gluconate.

12. A cosmetic composition comprising:
(a) 10%-25% of each of water, isododecane and trisiloxane;
(b) 5%-10% of trimethylsiloxysilicate;
(c) 1%-5% of each of cyclopentasiloxane, dimethicone crosspolymer, hexyl/succinyl dimethicone crosspolymer, dimethicone, acrylates copolymer, PEG-10 dimethicone, disteardimonium hectorite, glycerin, Pyrus malus fruit extract, and xylitylglucoside;
(d) 0.1-1% of each of sodium chloride, phenoxyethanol, anhydroxylitol, Glycine soja oil, xylitol, polyhydroxystearic acid, silica and glucose;
(e) 0%> but ≤0.5% of each of: Butyrospermum parkii butter; ethylhexylglycerin; aroma and flavor; Aloe barbadensis leaf juice; sodium hyaluronate; tocopheryl acetate; panthenol; butylene glycol; Calanthe discolor extract; urea; glucosamine HCL; Laminaria digitata extract; Saccharomyces cerevisiae extract; Camellia sinensis leaf extract; Coffea arabica seedcake extract; Euterpe oleracea fruit extract; Garcinia mangostana fruit extract; Lycium barbarum fruit extract; Morinda citrifolia fruit extract; Orchis mascula flower extract; Punica granatum fruit extract; tocopherol; hydrolyzed Opuntia ficus-indica flower extract; citric acid; xanthan gum; gluconolactone; 1,2-hexanediol; caprylyl glycol; sodium benzoate; and calcium gluconate; and
(f) one or more coloring agent.

13. A cosmetic composition consisting of:
(a) 10%-25% of each of water, isododecane and trisiloxane;
(b) 5%-10% of trimethylsiloxysilicate;
(c) 1%-5% of each of cyclopentasiloxane, dimethicone crosspolymer, hexyl/succinyl dimethicone crosspolymer, dimethicone, acrylates copolymer, CI 77891, PEG-10 dimethicone, CI 77491, disteardimonium hectorite, glycerin, Pyrus malus fruit extract, and xylitylglucoside;
(d) 0.1-1% of each of sodium chloride, phenoxyethanol, anhydroxylitol, Glycine soja oil, xylitol, CI 19140, polyhydroxystearic acid, CI 15850, silica and glucose; and
(e) 0%> but ≤0.5% of each of: Butyrospermum parkii butter; ethylhexylglycerin; CI 42090; aroma and flavor; Aloe barbadensis leaf juice; sodium hyaluronate; tocopheryl acetate; panthenol; butylene glycol; Calanthe discolor extract; urea; glucosamine HCL; Laminaria digitata extract; Saccharomyces cerevisiae extract; Camellia sinensis leaf extract; Coffea arabica seedcake extract; Euterpe oleracea fruit extract; Garcinia mangostana fruit extract; Lycium barbarum fruit extract; Morinda citrifolia fruit extract; Orchis mascula flower extract; Punica granatum fruit extract; tocopherol; hydrolyzed Opuntia ficus-indica flower extract; citric acid; xanthan gum; gluconolactone; 1,2-hexanediol; caprylyl glycol; sodium benzoate; and calcium gluconate.

14. The cosmetic composition of any one of claims 1-13, wherein the composition is for use on a user's lips.

15. The cosmetic composition of claim 14, wherein the composition provides one or more of the following when applied to the user's lips: resistance to smearing, non-drying, moisturizing, no discomfort, creation of unique colors by layering the composition, waterproof, long-lasting, gluten free, animal by-product free, and paraben free.

16. A cosmetic product comprising the composition of any one of claims 1-15.

17. The cosmetic product of claim 16, wherein the product is a lip cream, lip balm, lipstick or a lip-gloss.

18. The cosmetic product of claim 17, wherein the product provides one or more of the following when applied to a user: resistance to smearing, non-drying, moisturizing, no discomfort, creation of unique colors by layering the product, waterproof, long-lasting, gluten free, animal by-product free, and paraben free.

19. Use of the cosmetic composition of any one of claims 1-15 or the cosmetic product of any one of claims 16-18 for providing color to the lips of a user when the composition or product is applied.

20. The use of claim 19, wherein the composition is for a single application.

21. The use of claim 19, wherein the composition is for multiple applications.

22. The use of any one of claims 19-21, wherein the product, composition, or both provides one or more of the following when applied to the user: resistance to smearing, non-drying, moisturizing, no discomfort, creation of unique colors by layering, waterproof, long-lasting, gluten free, animal by-product free, and paraben free.

23. A method for obtaining a desired lip coloring and/or lip moisture comprising applying the composition of any one of claims 1-15 or the product of any one of claims 16-18 to a user's lips.

24. The method of claim 23, wherein the composition or product is applied to the user's lips one or more times to achieve the desired lip coloring and/or lip moisturization.

25. The method of claim 23 or 24, wherein the composition or product is long-lasting on the user's lips.

26. The method of any one of claims 23-25, wherein the composition or product is resistant to transfer from the user's lips.

27. The method of any one of claims 23-26, wherein the composition or product is waterproof on the user's lips.

28. The method of any one of claims 23-27, wherein the product, composition, or both provides one or more of the following when applied to the user: resistance to smearing, non-drying, moisturizing, no discomfort, creation of unique colors by layering, waterproof, long-lasting, gluten free, no animal by-products, and paraben free.

29. A composition, kit, method, use, product as described herein.
